(19) United States
(12) Patent Application Publication
(10) Pub. No.: US 2013/0236519 A1
(43) Pub. Date: Sep. 12, 2013

(54) Oral health care material and teeth cleaning agent composition

(71) Applicant: NATIONAL CHENG KUNG UNIVERSITY, Tainan City (TW)

(72) Inventors: Dar-Bin SHIEH, Tainan City (TW); Hsin-Ju CHANG, Kaohsiung City (TW); Hung-Ching LU, Tainan City (TW)

(73) Assignee: NATIONAL CHENG KUNG UNIVERSITY, Tainan City (TW)

(21) Appl. No.: 13/773,898

(22) Filed: Feb. 22, 2013

Related U.S. Application Data
(60) Provisional application No. 61/602,129, filed on Feb. 23, 2012.

(51) Int. Cl. A61K 8/27; A61K 8/02; A61Q 11/00

(52) U.S. Cl. CPC A61K 8/27; A61Q 11/00; A61K 8/0279; A61K 8/0245
USPC 424/401; 424/49; 428/402; 428/401; 428/398

(57) ABSTRACT

An oral health care material and a teeth cleaning agent composition are disclosed. The oral health care material is made of zinc oxide grains, where each is selected from the group consisting of zinc oxide crystalline nanograins, zinc oxide nanorods, zinc oxide hollow fibers, and a mixture thereof, wherein the diameter of each of the zinc oxide crystalline nanograins is 25 nm-200 nm, the cross-sectional diameter of each of the zinc oxide nanorods is 50 nm-1000 nm, the cross-sectional diameter of each of the zinc oxide hollow fibers is 500 nm-3 μm and its nanograin size is 20 nm-100 nm.

### CROSS REFERENCE TO RELATED APPLICATION

This application claims the benefit of filing date of U. S. Provisional Application Ser. No. 61/602,129, entitled “ZnO, Teeth Cleaning Composition, and Hard Tissue Regeneration Material Containing the Same” filed Feb. 23, 2012 under 35 USC §119(e)(1).

### BACKGROUND OF THE INVENTION

1. Field of the Invention

The present invention relates to an oral health care material and/or a teeth cleaning agent composition and, more particularly, to an oral health care material and/or a teeth cleaning agent composition comprising crystallized ZnO nanograins, crystallized ZnO nanorods, nano-ZnO hollow fibers, or a combination thereof.

2. Description of Related Art

Dental caries is a prevalent medical condition of the oral cavity, and remains one of the most frequently recorded diseases in clinical practice. Dental filling is the most commonly used medical procedure for the restoration of damaged dental structure as shown in dental caries. Since teeth are not capable of completely self-repair severe damages, spread of fissure caries can widen if the caries are not filled. General restoration treatment involves starting with removing septic matter in the cavities and using suitable material to fill the cavities, for the goal of preventing the cavity size from getting larger. Early treatment, in the case particularly when caries has just been detected, is desirable because such is less painful and easier to manage, as opposed to treatment performed when caries has reached the pulp.

In addition, oral cleaning is one important aspect of oral care. Oral cleaning can prevent not only dental caries but also periodontosis. Conventional oral cleaning is done by brushing the teeth and using products such as tooth paste, tooth powders and mouthwash, in order to improve the cleaning effect on teeth, remove dental plaque and prevent dental caries. Oral hygiene agents (including toothpaste, tooth powders and mouthwash) that contain extra inclusion of plaque-removing materials (for example, teeth cleaning agent compositions containing ZnO grains) are found to be effective for lowering caries formulation.

Hence, it is desirable to provide a filling material for dental cavities, or a teeth cleaning composition such as tooth paste, tooth powders and mouthwash, in order to improve the effect of dental caries prevention.

### SUMMARY OF THE INVENTION

The present invention provides an oral health care material containing zinc oxide (ZnO), where the zinc oxide is selected from the group consisting of crystallized ZnO nanograins, crystallized ZnO nanorods, nano-ZnO hollow fibers, and a combination thereof. The crystallized ZnO nanograins each has a diameter of 25 nm-200 nm, the cross-sectional diameter of each crystallized ZnO nanorod is 50 nm-1000 nm, the nano-ZnO hollow fibers each has a tube-like structure, and cross-sectional diameter of 500 nm-3 μm.

The ZnO of the present invention provides the following advantages: capability of decreasing or inhibiting the formation of dental caries; capability of decreasing or inhibiting gingivitis; capability of facilitating the curing of pains or wounds in mouths; capability of decreasing the level of acidogenic bacteria; capability of inhibiting the formation of biofilm in mouths; capability of decreasing the accumulation of dental plaque; capability of decreasing the dental erosion; and/or capability of facilitating the cleaning of teeth and mouths.

In the oral health care material of the present invention, the nano-ZnO hollow fibers each is preferably composed of a number of ZnO nanograins, and the diameter thereof is preferably 20 nm-100 nm. More specifically, each nano-ZnO hollow fiber preferably has a poly-crystalline structure.

In the oral health care material of the present invention, preferably, each of the crystallized ZnO nanograins is selected from the group consisting of single-crystallized ZnO grains, poly-crystallized ZnO grains, and a combination thereof.

In the oral health care material of the present invention, the preferred diameter of the crystallized ZnO nanograin is 50 nm-100 nm.

In the oral health care material of the present invention, the cross-sectional diameter of the crystallized ZnO nanorod is preferably 200 nm-500 nm.

The present invention further provides a teeth cleaning agent composition, which comprises an orally acceptable excipient and ZnO nanograins selected from the group consisting of crystallized ZnO nanograins, crystallized ZnO nanorods, nano-ZnO hollow fibers, and a combination thereof, wherein the content ratio of the ZnO nanograins is 10-60 wt % based on a total weight of the teeth cleaning agent composition, Herein, diameter of each crystallized ZnO nanograin is 25 nm-200 nm, cross-sectional diameter of each crystallized ZnO nanorod is 50 nm-1000 nm, the nano-ZnO hollow fiber has a tube-like structure, and the cross-sectional diameter of each nano-ZnO hollow fiber is 500 nm-3 μm.

The teeth cleaning agent composition of the present invention can be any composition for cleaning teeth such as tooth paste, tooth powder and mouthwash. In addition, the present invention also benefits from the inclusion of crystalline ZnO nanograins to see increased bacteria-killing performance, as well as oral cavity cleaning performance. Furthermore, because the ZnO nanograin contained in the teeth cleaning agent composition of the present invention has specific photo-catalytic property and a nanoscale structure, the teeth cleaning composition is enabled with intrinsic antibacterial capability and deep interstitial plaque-cleaning capability. These advantageous effects, as a result, are contributive to the improved oral cavity cleaning.

In addition, any other components may be further added as additives into the teeth cleaning agent composition, examples of the additives include desensitizers such as potassium nitrate, whitening agents such as hydrogen peroxide, calcium peroxide and strontium peroxide, preservatives, silicone and chlorophyll compound. The presence of these additives in the teeth cleaning agent composition will not substantially affect the nature and characteristics expected for the teeth cleaning agent composition.

In the teeth cleaning agent composition, the orally acceptable excipient can be water and wetting agents. Usually, the orally acceptable excipient is a mixture of water, wetting agents and alcohol. The alcohol is chosen for non-toxic alcohol, such as ethanol or isopropanol. The content of the wetting agents, which include glycerol, sorbic acid, sodium lauryl sulfate, mineral oil, polyethylene or alkane diol (for example, ethylene glycol or propylene glycol) can be about 10-30 wt %. When the teeth cleaning agent composition of the present invention is used as mouthwash, its content would comprise more than about 45 wt % of water. Preferably, the mouthwash comprises about 50-85 wt % of water, about 0-20 wt % of non-toxic alcohol, and about 10-40 wt % of wetting agents.

The teeth cleaning agent composition of the present invention may further comprise a flavoring agent. The flavoring agent to be used in the present invention may be essential oils, aromatic aldehydes, esters, alcohols or similar substances. Examples of the essential oils include oils of spearmint, mint, wintergreen, sassafras, clove, salvia, eucalyptus, marjoram, cinnamon, lemon, lime, grape and orange.

In the teeth cleaning agent composition of the present invention, preferably, the nano-ZnO hollow fibers are composed of plural ZnO nanograins.

In the teeth cleaning agent composition of the present invention, preferably, the diameter of the aforementioned ZnO nanograin is 20 nm-100 nm.

In the teeth cleaning agent composition of the present invention, the crystallized ZnO nanograin is preferably selected from the group consisting of single-crystallized ZnO grains, poly-crystallized ZnO grains, and a combination thereof.

In addition, in the teeth cleaning agent composition of the present invention, the crystallized ZnO nanograins each has a preferred diameter of 50 nm-100 nm.

Furthermore, in the teeth cleaning agent composition of the present invention, the preferred cross-sectional diameter of the crystallized ZnO nanorod is 200 nm-500 nm.

### BRIEF DESCRIPTION OF THE DRAWINGS

FIG. 1 are X-ray diffraction (XRD) patterns according to testing example 1 of the present invention;

FIGS. 2A-2C are field emission scanning electron microscopy (FE-SEM) photos according to testing example 2 of the present invention;

FIG. 2D is a transmitting electron microscopy (TEM) photo according to testing example 3 of the present invention;

FIGS. 3A-3C are photoluminescence spectra according to testing example 4 of the present invention; and

FIG. 4 is a result showing the anti-bacteria property according to testing example 5 of the present invention.

### DETAILED DESCRIPTION OF THE PREFERRED EMBODIMENT

### Embodiment 1

### Preparation of ZnO Nanorods

In the present embodiment, a chemical bath deposition method was used to prepare ZnO nanorods, and the process thereof is shown as follows. 0.1 M of Zn(NO₃)₂ solution (available from Aldrich) was added into 0.1 M hexamethyleneteramine (HMTA) solution and the mixture was stirred well. During stirring, white precipitates were formed due to ZnO nucleation. Next, the mixture was placed in an oven at 95° C. for 8 hr to form ZnO crystals. After the crystals were formed, the un-reacted starting materials were removed by a centrifuge at 3500 rpm for 10 min. The obtained ZnO nanorods were sequentially washed with distilled water and ethanol. After drying, ZnO nanorods of the present embodiment were obtained, and the cross-sectional diameter for each nanorod was about 200 nm-500 nm.

### Embodiment 2

### Preparation of ZnO Hollow Fibers

In the present embodiment, a template-based method was used to prepare ZnO hollow fibers, and the process thereof is shown as follows. Cotton fibers (Consumed, 5 cm×5 cm) were used as templates, and dipped into a 3.5 wt % of zinc acetate containing solution (available from JTBaker) to coat the cotton fibers with zinc acetate. Next, the products were placed at 50° C. for 2 hr to remove water, in order to decrease the formation of voids. Then, the cotton fibers coated with zinc acetate were placed in an oven at 600° C., sintered under air atmosphere for 2 hr, and slowly cooled to room temperature. During sintering process, the cotton fibers were degraded into CO₂, water or other small hydrocarbon molecules, and the zinc acetate was transferred into zinc oxide (ZnO). After the aforementioned process, nano-ZnO hollow fibers of the present embodiment can be obtained.

The obtained nano-ZnO hollow fibers of the present embodiment had a poly-crystalline structure, and more specifically the obtained nano-ZnO hollow fibers of the present embodiment was composed of plural ZnO nanograins, each having a diameter of 20 nm-100 nm.

### Embodiment 3

### Preparation of ZnO Nanograins

The nano-ZnO hollow fibers were pounded into pieces to obtain plural ZnO nanograins, in which each has a diameter of about 50 nm-100 nm.

### Embodiment 4

The teeth cleaning agent composition in this example is used as a tooth paste, and the process for preparing the same is shown as follows. First, 1.75 g of sodium carboxymethyl cellulose (available from CHENG YI CHEMICAL CO., LTD.) was added into 7 mL of water, and the obtained solution was stirred until the appearance thereof was present in a transparent gel state. 20 g of polyethylene glycol (PEG 400, available from J.T.BAKER), 8.2 g of glycerol (available from wako), 0.02 g of methyl benzoate (available from CHENG YI CHEMICAL CO., LTD.), and 2 g of sodium lauryl sulfate (available from Sigma) was added into 1 mL of water to obtain a mixture. The well-stirred mixture was added into the prepared solution of sodium carboxymethyl cellulose, and stirred. The obtained mixture was placed into a mortar, and then 10 g of crystallized ZnO nanorods and 40 g of calcium carbonate was slowly added therein while the mixture was stirred and ground. Finally, 1 g of mineral oil (available from Showa) was added therein, and water was then added therein to make the final volume of the mixture, which was 100 mL.

In the present embodiment, the crystallized ZnO nanorods can be selectively substituted with ZnO hollow fibers obtained from Embodiment 2 or ZnO nanograins obtained from Embodiment 3.

Alternatively, other components may also be added into the teeth cleaning agent composition of the present embodiment, and the examples thereof include desensitizers such as potassium nitrate, whitening agents such as hydrogen peroxide, calcium peroxide and strontium peroxide, preservative, silicone and chlorophyll compound. The presence of these additives in the teeth cleaning agent composition will not substantially affect the nature and characteristics expected for the teeth cleaning agent composition.

In the present embodiment, the content of the wetting agents such as glycerol, sorbic acid, polyethylene or alkane diol (for example, ethylene glycol or propylene glycol) can be 1-10 wt %. For example, when the teeth cleaning agent composition of the present embodiment is used as mouthwash, it can comprise more than about 45 wt % of water. Preferably, the mouthwash comprises about 50-85 wt % of water, about 0-20 wt % of non-toxic alcohol, and about 10-40 wt % of wetting agents.

In addition, the teeth cleaning agent composition of the present embodiment may further comprise flavoring agents, for which examples include essential oils, aromatic aldehydes, esters, alcohols or similar substances.

Furthermore, when the teeth cleaning agent composition of the present embodiment is used as mouthwash, it may further comprise an alcohol, and the examples thereof can be non-toxic alcohol such as ethanol or isopropanol.

### Testing Example 1

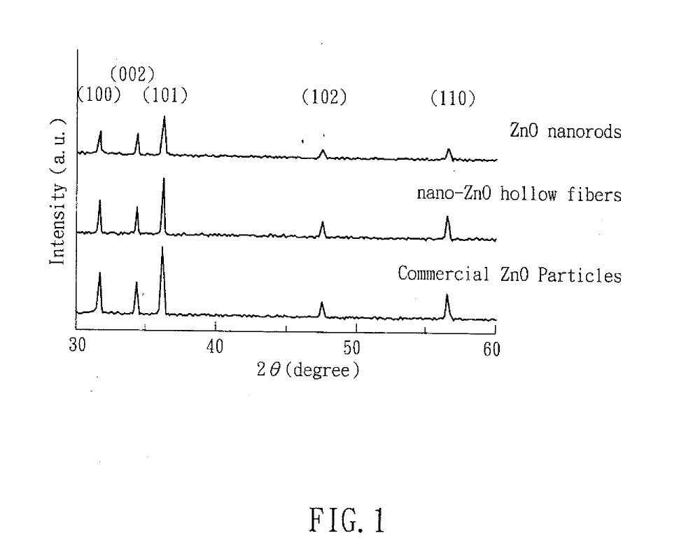

Crystal structures of commercial ZnO grains (available from SHOFU, HY-Bond), the ZnO nanorods prepared in Embodiment 1 and the nano-ZnO hollow fibers prepared in Embodiment 2 were analyzed by X-ray diffraction, which was performed with Rigaku D/MAX-2000. The obtained results are shown in FIG. 1.

According to the XRD patterns shown in FIG. 1, the data obtained from these ZnO grains are similar to that of the standard of hexagonal wurtzite structure (JCPDS, 36-1451). In addition, as shown in FIG. 1, there are no contamination peaks found in the patterns of the synthetic ZnO grains of the present invention (i.e. ZnO nanorods and nano-ZnO hollow fibers). These results indicate that the synthetic ZnO grains of the present invention have very high purity.

### Testing Example 2

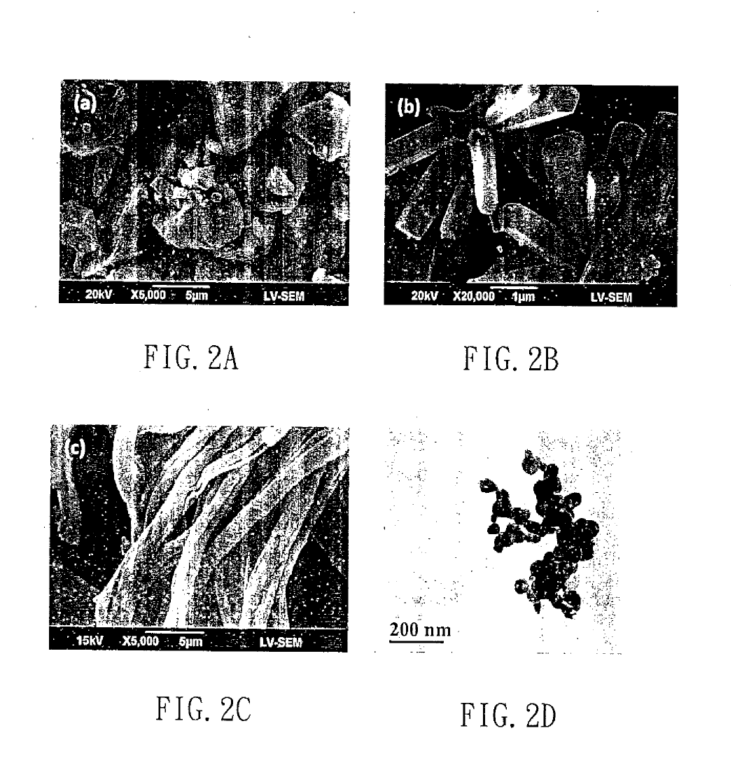

The commercial ZnO grains (available from SHOFU, HY-Bond), the ZnO nanorods prepared in Embodiment 1 and the nano-ZnO hollow fibers prepared in Embodiment 2 were analyzed with field emission scanning electron microscopy (FE-SEM, JEOL JSM-7000F), and the obtained photos are shown in FIGS. 2A-2C.

As shown in FIG. 2A, the size of each commercial ZnO grain is about several micro-meters, and the shape thereof is variable.

However, as shown in FIG. 2B, the synthetic ZnO nanorod according to Embodiment 1 of the present invention has a hexagonal structure, and the diameter thereof is about 200 nm-500 nm for each nanorod.

In addition, as shown in FIG. 2C, the shape of the synthetic nano-ZnO hollow fiber is the same as that of the cotton fiber, and the diameter thereof is about 1 μm-2 μm.

### Testing Example 3

The composed nano-ZnO hollow fibers prepared in Embodiment 2 were analyzed with transmission electron microscopy (TEM, JEOL 2010), and the result thereof is shown in FIG. 2D.

As shown in FIG. 2D, the nano-ZnO hollow fibers are composed of many ZnO nanograins, and the diameters of these ZnO nanograins are each about 50 nm-100 nm.

### Testing example 4

The commercial ZnO grains (available from SHOFU, HY-Bond), the

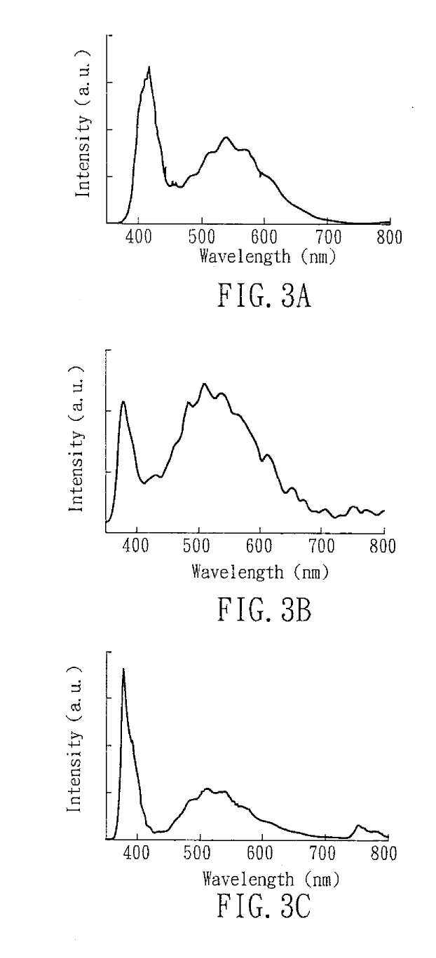

ZnO nanorods prepared in Embodiment 1 and the nano-ZnO hollow fibers prepared in Embodiment 2 were analyzed with a fluorescence spectrometer (type: Perkin-Elmer LS55), wherein xenon laser with a wavelength of 325 nm was used as an excitation source, and photoluminescence spectra of ZnO grains were measured. The results are shown in FIGS. 3A-3C, wherein FIG. 3A is a spectrum of a ZnO nanorod, FIG. 3B is that of a nano-ZnO hollow fiber, and FIG. 3C is that of a commercial ZnO grain.

FIGS. 3A-3C show a UV emission band near 375 nm and a green emission band near 530 nm. The green emission band is mainly a result stemming from the photons released from the electron transition between photogenerated holes and positively charged oxygen vacancies. The intensity of green band near 530 nm varies with the size of ZnO material. More specifically, the intensity of green band may increase, and in some situations by even a higher degree than that of the UV emission in the case when the size of ZnO material is downgraded to the nano-scale. In addition, these bands of ZnO nanorods exhibit a red-shift spectrum; this may be a result of the disproportion between nanorod shape and the sphere.

### Testing example 5

### Anti-bacteria Property Test

The commercial ZnO grains (available from SHOFU, HY-Bond), the ZnO nanorods prepared in Embodiment 1 and the nano-ZnO hollow fibers prepared in Embodiment 2 were analyzed by a micro-MIC test. First, Streptococcus mutans (ATCC 25175) were activated overnight and transferred into 96-wells plate. The initial OD_{600 nm} of bacteria was 0.03. The standard bacteria solution was grown to mid-log phase. Then, a suitable amount of ZnO grains was added into the nutrient broth to make the final concentration of ZnO in each well (1000 μl) about 0.05 mg/ml and 0.1 mg/ml. The nutrient broth without ZnO was used as a control group, and the nutrient broth with ZnO having identical concentration but without bacteria was used as a blank group. After the bacteria was cultured at 37° C. for 4 hr, the 96-wells plate was analyzed with an ELISA reader (TECAN, sunrise) under a wavelength of 600 nm. MIC50 was considered as a lower concentration to inhibit 50% of bacteria, and the obtained results were compared with that of the control group without adding ZnO.

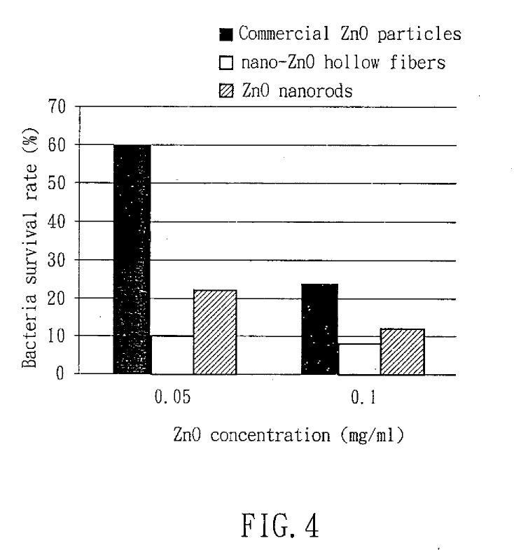

The results are shown in FIG. 4. The inhibiting effects of the crystallized ZnO nanorods and the nano-ZnO hollow fibers are significantly better than that of the commercial ZnO grains when the concentration of ZnO is 0.05 mg/ml or more. These results indicate that the crystallized ZnO nanorods or the nano-ZnO hollow fibers of the present invention can decrease or inhibit the formation of dental caries and further improve the effect of cavity protection when they are used in the oral health care material or the teeth cleaning agent composition.

In conclusion, the crystallized ZnO nanograins, crystallized ZnO nanorods, nano-ZnO hollow fibers or a combination thereof contained in the oral health care material and/or the teeth cleaning agent composition of the present invention have specific photo-catalytic properties and are of a nanoscale size, so the oral health care material and/or the teeth cleaning agent is/are enabled with intrinsic antibacterial capability and deep interstitial plaque-cleaning capability. Moreover, the ZnO grains of the present invention have better bactericidal properties. Hence, the oral health care material and/or the teeth cleaning agent composition of the present invention can show better effects than those contained conventional ZnO grains with large diameters.

Although the present invention has been explained in relation to its preferred embodiment, it is to be understood that many other possible modifications and variations can be made without departing from the spirit and scope of the invention as hereinafter claimed.

What is claimed is:

1. An oral health care material which comprises zinc oxide (ZnO) grains, each zinc oxide grain is selected from the group consisting of crystallized ZnO nanograins, crystallized ZnO nanorods, nano-ZnO hollow fibers, and a combination thereof;
wherein each of the crystallized ZnO nanograins has a diameter of 25 nm-200 nm, each of the crystallized ZnO nanorods has a cross-sectional diameter of 50 nm-1000 nm, each of the nano-ZnO hollow fibers has a tube-like structure, and each of the nano-ZnO, hollow fibers has a cross-sectional diameter of 500 nm-3 μm.

2. The oral health care material as claimed in claim I, wherein the each of the nano-ZnO hollow fibers is composed of plural ZnO nanograins.

3. The oral health care material as claimed in claim 2, wherein the each ZnO nanograin has a diameter of 20 nm-100 nm.

4. The oral health care material as claimed in claim 1, wherein the each of the crystallized ZnO nanograins is selected from the group consisting of single-crystallized ZnO grains, poly-crystallized ZnO grains, and a combination thereof.

5. The oral health care material as claimed in claim 1, wherein the each of the crystallized ZnO nanograins has a diameter of 50 nm-100 nm.

6. The oral health care material as claimed in claim 1, wherein the each of the crystallized ZnO nanorods has a cross-sectional diameter of 200 nm-500 nm.

7. A teeth cleaning agent composition comprising:
an orally acceptable excipient; and
ZnO grains selected from the group consisting of: crystallized ZnO nanograins, crystallized ZnO nanorods, nano-ZnO hollow fibers, and a combination thereof, wherein a content of the ZnO grains is 10-60 wt % based on a total weight of the teeth cleaning agent composition,
wherein each of the crystallized ZnO nanograins has a diameter of 25 nm-200 nm, each of the crystallized ZnO nanorods has a cross-sectional diameter of 50 nm-1000 nm, each of the nano-ZnO hollow fibers has a tube-like structure, and each of the nano-ZnO hollow fibers has a cross-sectional diameter of 500 nm-3 μm.

8. The teeth cleaning agent composition as claimed in claim 7, wherein the each of the nano-ZnO hollow fibers comprises a plurality of ZnO nanograins.

9. The teeth cleaning agent composition as claimed in claim 8, wherein the each of the plurality of ZnO nanogram has a diameter of 20 nm-100 nm.

10. The teeth cleaning agent composition as claimed in claim 7, wherein the each of the crystallized ZnO nanograins is selected from the group consisting of single-crystallized ZnO grains, poly-crystallized ZnO grains, and a combination thereof.

11. The teeth cleaning agent composition as claimed in claim 7, wherein the each of the crystallized ZnO nanograins has a diameter of 50 nm-100 nm.

12. The teeth cleaning agent composition as claimed in claim 7, wherein the each of the crystallized ZnO nanorods has a cross-sectional diameter of 200 nm-500 nm.
